(19) United States
(12) Patent Application Publication
(10) Pub. No.: US 2023/0060422 A1
(43) Pub. Date: Mar. 2, 2023

(54) COMBINATION TREATMENT OF LIVER DISEASES USING INTEGRIN INHIBITORS

(71) Applicants: NOVARTIS AG, Basel (CH); PLIANT THERAPEUTICS, INC., South San Francisco (US)

(72) Inventors: Kraig ANDERSON, South San Francisco, CA (US); Christopher BAILEY, South San Francisco, CA (US); Avirup BOSE, Marlborough, MA (US); Jacob CHA, South San Francisco, CA (US); Nicole COOPER, Oakland, CA (US); Darren FINKELSTEIN, South San Francisco, CA (US); Linda GREENBAUM, Florham Park, NJ (US); Johannes HULL, San Francisco, CA (US); Susan Caroline KIRKLAND, Surrey (GB); Katerina LEFTHERIS, San Mateo, CA (US); Maureen REILLY, Burlingame, CA (US); Peter TARSA, Marshfield, MA (US); Chinweike UKOMADU, Chestnut Hill, MA (US)

(21) Appl. No.: 17/786,491

(22) PCT Filed: Dec. 18, 2020
(86) PCT No.: PCT/US2020/066053

§ 371 Date: Jun. 16, 2022

Related U.S. Application Data
(60) Provisional application No. 62/951,600, filed on Dec. 20, 2019.

(51) Int. Cl. A61K 31/4162; A61K 31/4375; A61K 31/7048; A61K 45/06; A61P 1/16

(52) U.S. Cl. CPC A61K 31/4162; A61K 31/4375; A61K 31/7048; A61K 45/06; A61P 1/16

(57) ABSTRACT

The invention provides a pharmaceutical combination including an αvβ₁ integrin inhibitor and at least one additional therapeutic agent for preventing, delaying or treating liver diseases or disorders. The additional therapeutic agent may be an SGLT1/2 inhibitor, among a diverse selection of agents. For example, the pharmaceutical combination includes (S)-2-(4-methyltetrahydro-2H-pyran-4-carboxamido)-9-(5,6,7,8-tetrahydro-1,8-naphthyridin-2-yl) nonanoic acid (Compound 1) and at least one additional therapeutic agent.

### FIELD OF THE INVENTION

The present invention relates to a combination therapy for treating, preventing, or ameliorating conditions mediated by a fibrotic integrin and at least one additional therapeutic agent, in particular liver diseases, comprising administering to a subject in need thereof a therapeutically effective amount of an integrin inhibitor and at least one additional therapeutic agent. Furthermore, the present invention is directed to a pharmaceutical combination comprising an α_{V}β₁ integrin inhibitor and at least one additional therapeutic agent, optionally in the presence of a pharmaceutically acceptable carrier, and pharmaceutical compositions comprising them.

### BACKGROUND OF THE INVENTION

Nonalcoholic fatty liver disease (NAFLD) is the most common cause of chronic liver disease in the Western world. NAFLD is a chronic liver disease (CLD) that was long thought to be a non-progressive form of fatty liver. However, recent clinical and preclinical evidence indicates that NAFLD can progress to more severe non-alcoholic steatohepatitis (NASH) and, as a consequence, patients can develop hepatic fibrosis where there is a persistent inflammation in the liver resulting in the generation of fibrous scar tissue around the liver cells and blood vessels. Eventually, cirrhosis can develop over time whose damage is permanent and can lead to liver failure and liver cancer (hepatocellular carcinoma). Thus, the main stages of NAFLD are: 1) simple fatty liver (steatosis); 2) NASH; 3) fibrosis; and 4) cirrhosis.

Liver transplantation is the only treatment for advanced cirrhosis with liver failure. Estimates of the worldwide prevalence of NAFLD range from 6.3% to 33% with a median of 20% in the general population. The estimated prevalence of NASH is lower, ranging from 3 to 5% (Younossi et al., Hepatology, Vol. 64, No. 1, 2016). NASH is a worldwide problem with growing prevalence over the last few decades. Over the last decade NASH has risen from uncommon to the second indication for liver transplantation in the US. It is expected to be the leading cause of transplant by 2024. NASH is highly associated with the metabolic syndrome and Type 2 diabetes mellitus. Furthermore, cardiovascular mortality is an important cause of death in NASH patients.

Development of NASH involves several mechanisms: accumulation of fat in the liver (steatosis), inflammation of the liver, hepatocyte ballooning, and fibrosis. The NAFLD Activity Score (NAS) was developed as a tool to measure changes in NAFLD during therapeutic trials. The score is calculated as the unweighted sum of the scores for steatosis (0-3), lobular inflammation (0-3), and ballooning (0-2).

In chronic liver diseases such as NASH, activated hepatic stellate cells account for the major source of myofibroblasts that drive fibrogenesis (Higashi et al. 2017), and transforming growth factor beta (TGF-β) is a major driver of myofibroblast activation. TGFβ1 is initially secreted with the latency-associated peptide (LAP) that keeps TGFβ1 in an inactive state. One method by which TGF-β1 is converted into its active form is through an interaction between LAP and the α_{V} integrins, including a_{V}β₁. The α_{V}β₁ integrin is an RGD-binding integrin expressed on fibroblasts, and is thought to significantly contribute to TGF-β1 activation in fibrotic liver tissue (Parola et al. 2008, Reed et al. 2015). Pharmacologic inhibition of α_{V}β₁ has been shown to decrease fibrosis in a mouse model of liver fibrosis (Reed et al. 2015). As TGF-β1 signaling is involved in a wide variety of homeostatic processes throughout the body, it is believed that inhibition of the α_{V}β₁TGF-β1 axis specific to fibrotic tissues may allow for a localized, and therefore potentially safer, targeting of TGFβ1 signaling (Henderson et al. 2013, Henderson and Sheppard 2013, Reed et al. 2015).

Farnesoid X Receptor (FXR) is a nuclear receptor activated by bile acids, also known as Bile Acid Receptor (BAR). FXR is expressed in principal sites of bile acid metabolism, such as liver, intestine and kidney, where it mediates effects on multiple metabolic pathways in a tissue-specific manner.

The mode of action of FXR in the liver and intestine is well known, and is described e.g. in Calkin and Tontonoz, (2012) (Nature Reviews Molecular Cell Biology 13, 213-24). FXR is responsible for modulating bile acid production, conjugation and elimination through multiple mechanisms in the liver and intestine. In normal physiology, FXR detects increased levels of bile acids and responds by decreasing bile acid synthesis and bile acid uptake while increasing bile acid modification and secretion in the liver. In the intestine, FXR detects increased bile acid levels and decreases bile acid absorption and increases secretion of FGF15/19. The net result is a decrease in the overall levels of bile acids. In the liver, FXR agonism increases expression of genes involved in canalicular and basolateral bile acid efflux and bile acid detoxifying enzymes while inhibiting basolateral bile acid uptake by hepatocytes and inhibiting bile acid synthesis.

Furthermore, FXR agonists decrease hepatic triglyceride synthesis leading to reduced steatosis, inhibit hepatic stellate cell activation reducing liver fibrosis, and stimulate FGF15/FGF19 expression (a key regulator of bile acid metabolism) leading to improved hepatic insulin sensitivity. Thus, FXR acts as a sensor of elevated bile acids and initiates homeostatic responses to control bile acid levels, a feedback mechanism that is believed to be impaired in cholestasis. FXR agonism has shown clinical benefits in subjects with cholestatic disorders (Nevens et al., J. Hepatol. 60 (1 SUPPL. 1): 347A-348A (2014)), bile acid malabsorption diarrhea (Walters et al., Aliment Pharmacol. Ther. 41(1):54-64 (2014)) and non-alcoholic steatohepatitis (NASH; Neuschwander-Tetri et al 2015). The FXR agonist Nidufexor (LMB763) is currently being evaluated in NASH patients with fibrosis.

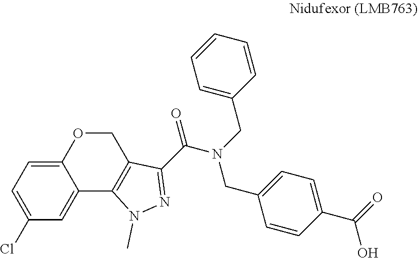 [SMILES: Cn1nc(C(=O)N(Cc2ccccc2)Cc2ccc(C(=O)O)cc2)c2c1-c1cc(Cl)ccc1OC2]

In addition, the following classes of compounds or therapeutics have been explored to mediate metabolic dysfunctions: glucagon-like peptide 1 (GLP-1) receptor agonists (GLP-1RAs) and dipeptidyl peptidase-4 (DPP4) inhibitors, peroxisome proliferator-activated receptor (PPARs) agonists, acetyl-CoA carboxylase (ACC) inhibitors, thyroid hormone receptor β (TRβ) agonists, ketohexokinase (KHK) inhibitors, diacylglycerol Acyltransferase 2 (DGAT2) inhibitors, and sodium-glucose linked transporter (SGLT) inhibitors.

Other related targets and agents include: anti-inflammatory agents (such as chemokine receptor 2/5 (CCR2/5) antagonists), and anti-fibrosis agents (such as Galectin-3 inhibitors and Lysyl oxidase-like 2 (LOXL 2) inhibitors).

Because the pathophysiology of NAFLD and NASH is complex and multiple redundant pathways may be implicated, there is a need to provide treatments for NAFLD, NASH and fibrotic/cirrhotic that can address the different aspects of these complex conditions, while demonstrating an acceptable safety and/or tolerability profile.

### SUMMARY OF THE INVENTION

The invention provides a pharmaceutical combination comprising, separate or together, at least an α_{V}β₁ integrin inhibitor and at least one additional therapeutic agent, for simultaneous, sequential, or separate administration. The invention further provides a medicament comprising the pharmaceutical combination.

In one aspect, the α_{V}β₁ integrin inhibitor is (S)-2-(4-methyltetrahydro-2H-pyran-4-carboxamido)-9-(5,6,7,8-tetrahydro-1,8-naphthyridin-2-yl)nonanoic acid (Compound 1, shown below), a stereoisomer, a tautomer, an enantiomer, a pharmaceutically acceptable salt, a prodrug, an ester thereof or an amino acid conjugate thereof.

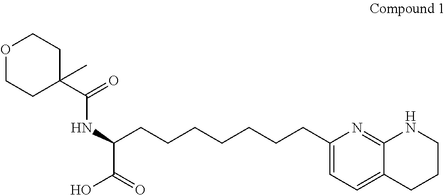 [SMILES: CC1(C(=O)N[C@@H](CCCCCCCc2ccc3c(n2)NCCC3)C(=O)O)CCOCC1]

In another aspect, the at least one additional therapeutic agent is selected from the group consisting of: an FXR agonist (e.g., M480 (Metacrine), NTX-023-1 (Ardelyx), INV-33 (Innovimmune), and obeticholic acid), Steroyl-CoA desaturase-1 (SCD-1) inhibitor (e.g., arachidyl amido cholanoic acid (Aramchol™)), THR-β agonist (e.g., MGL-3196 (Resmetirom), VK-2809, MGL-3745 (Madrigal)), galectin-2 inhibitor (e.g., GR-MD-02/Belapectin), PPAR agonist (e.g., saroglitazar, seladelpar, elafibranor, lanifibranor, lobeglitazone, pioglitazone, IVA337 (Inventiva), CER-002 (Cerenis), MBX-8025 (Seladelpar)), GLP-1 agonist (e.g., exenatide, liraglutide, semaglutide, NC-101 (Naia Metabolic), G-49 (Astrazeneca), ZP2929 (BI/Zealand), PB-718 (Peg Bio)), FGF agonist (e.g., pegbelfermin (ARX618), BMS-986171, NGM-282, NGM-313, YH25724, and proteins disclosed in WO2013049247, WO2017021893 and WO2018146594), tirzepatide, pyruvate synthase inhibitors (e.g., nitazoxanide), Apoptosis signal-regulating kinase 1 (ASK1) inhibitor (e.g., selonsertib (GS-4997), GS-444217), Acetyl-CoA carboxylase (ACC) inhibitor (e.g., firsocostat (GS-0976), PF-05221304, gemcabene (Gemphire)), CCR inhibitor (e.g., AD-114 (AdAlta), Bertilimumab (Immune), CM-101 (ChemomAb), CCX-872 (ChemoCentryx), Cenicriviroc), thiazolidinedione (e.g, MSDC-0602K, Pioglitazone), sodium-glucose co-transporter-2 and 1 (SGLT1/2) inhibitor (e.g., Remogliflozin, luseogliflozin, dapagliflozin, licogliflozin), DPP-4 inhibitor (sitagliptin, saxagliptin, vildagliptin, linagliptin, evogliptin, gemigliptin, anagliptin, teneligliptin, alogliptin, trelagliptin, omarigliptin, gosogliptin, dutogliption), insulin receptor agonist (e.g. ORMD 0801 (Oramed)), SGLT-2 inhibitor with DPPP inhibitor (e.g. empagliflozin and linagliptin), insulin sensitizer (e.g., MSDC-0602K (Octeta/Cirius)), CCR2/5 inhibitor (e.g., CVC (Allergan), anti-BMP9 antibodies (e.g., the antibodies described in WO2016193872); a compound selected from the group consisting of ((R)-3-amino-4-(5-(4-((5-chloropyridin-2-yl)oxy)phenyl)-2H-tetrazol-2-yl)butanoic acid; (R)-3-amino-4-(5-(4-((5-chloro-3-fluoropyridin-2-yl)oxy)phenyl)-2H-tetrazol-2-yl)butanoic acid; (R)-3-amino-4-(5-(3-((5-(trifluoro-methyl)pyridin-2-yl)oxy)phenyl)-2H-tetrazol-2-yl)butanoic acid; (R)-3-amino-4-(5-(4-((5-(trifluoro-methyl)pyridin-2-yl)oxy)phenyl)-2H-tetrazol-2-yl)butanoic acid; (S)-3-amino-4-(5-(4-((5-chloro-3-fluoropyridin-2-yl)oxy)phenyl)-2H-tetrazol-2-yl)butanoic acid; (R)-3-amino-4-(5-(4-phenethoxyphenyl)-2H-tetrazol-2-yl)butanoic acid; and (R)-3-amino-4-(5-(4-(4-chlorophenoxy)-phenyl)-2H-tetrazol-2-yl)butanoic acid; or a pharmaceutically acceptable salt thereof, or any combination thereof.

In another aspect, the combination is a fixed dose combination.

In another aspect, the combination is a free combination.

In another aspect, the α_{V}β₁ integrin inhibitor and the at least one additional therapeutic agent can be administered together, one after the other, separately, in one combined unit dose form, or in two separate unit dose forms. The unit dose form may also be a fixed combination.

In another aspect, the pharmaceutical combination is used in the manufacture of a medicament for preventing, delaying or treating a liver disease or disorder.

In one aspect, the invention relates to such pharmaceutical combinations, e.g. fixed or free combinations, e.g. combined unit doses, for use in treating, preventing or treating a fibrotic or cirrhotic disease or disorder, e.g. a liver disease or disorder. In some aspects, such pharmaceutical combination comprises an α_{V}β₁ integrin inhibitor, e.g. Compound 1, and the at least one additional therapeutic agent, each being in an amount that is jointly therapeutically effective. In another aspect, the at least one additional therapeutic agent is a non-bile acid derived farnesoid X receptor (FXR) agonist. The non-bile acid derived FXR agonist is nidufexor.

The invention provides the use of an α_{V}β₁ integrin inhibitor, e.g. Compound 1 in combination with the at least one additional therapeutic agent, e.g. fixed or free combination, for the manufacture of a medicament for the prevention or treatment of a liver disease or disorder, e.g. a chronic liver disease or disorder, e.g cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis, e.g. NAFLD, NASH, hepatic fibrosis, hepatosteatosis or PBC.

In some aspects of the invention, the invention provides a method of preventing, delaying or treating a liver disease or disorder, in a patient in need therefor, comprising the step of administering a therapeutically effective amount of 1) an α_{V}β₁ integrin inhibitor, e.g. Compound 1, and 2) at least one additional therapeutic agent, e.g. an SGLT inhibitor (e.g. SGLT1/2 inhibitor, e.g. licogliflozin, dapagliflozin, canagliflozin, empagliflozin, ipragliflozin, ertugliflozin, and mizagliflozin), an FGF21 analog (e.g. pegbelfermin (BMS-986036) and BMS-986171), an FGF19 analog (e.g. aldafermin), thyroid hormone receptor β (THRβ) agonist (e.g. resmetirom (MGL-3196) and BMS-986171), a DPP4 inhibitor (e.g. sitagliptin), or an FXR agonist (e.g. obeticholic acid), wherein the liver disease or disorder is a chronic liver disease or disorder, e.g cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis, e.g. NAFLD, NASH, hepatic fibrosis, hepatosteatosis or PBC.

In some aspects of the invention, the invention provides a pharmaceutical combination for use in preventing, delaying or treating a chronic liver disease or disorder, e.g. NAFLD, NASH, hepatosteatosis, liver fibrosis, cirrhosis, PBC, and steatosis.

In some aspects of the invention, the invention provides a pharmaceutical combination for use in preventing, delaying or treating NASH, the pharmaceutical combination comprising 1) an α_{V}β₁ integrin inhibitor and 2) at least one additional therapeutic agent.

In some aspects of the invention, the invention provides a pharmaceutical combination for use in preventing, delaying or treating liver fibrosis, the pharmaceutical combination comprising 1) an α_{V}β₁ integrin inhibitor and 2) at least one additional therapeutic agent.

In some aspects of the invention, the invention provides a pharmaceutical combination for use in preventing, delaying or treating hepatosteatosis, the pharmaceutical combination comprising 1) an α_{V}β₁ integrin inhibitor and 2) at least one additional therapeutic agent.

In some aspects of the invention, the invention provides a pharmaceutical combination for use in preventing, delaying or treating hepatocellular ballooning, the pharmaceutical combination comprising 1) an α_{V}β₁ integrin inhibitor and 2) at least one additional therapeutic agent.

In some aspects of the invention, the invention provides a pharmaceutical combination for use in preventing, delaying or treating PBC, the pharmaceutical combination comprising 1) an α_{V}β₁ integrin inhibitor and 2) at least one additional therapeutic agent.

The invention provides a method of preventing, delaying or treating a liver disease or disorder, in a patient in need therefor, comprising administering a therapeutically effective amount of each active ingredient in the pharmaceutical combination of the current invention, the pharmaceutical combination comprising 1) an α_{V}β₁ integrin inhibitor and 2) at least one additional therapeutic agent. The liver disease or disorder is a fibrotic or cirrhotic liver disease or disorder, e.g. a liver disease or disorder, e.g. a chronic liver disease or disorder, e.g. NAFLD, NASH, liver fibrosis, cirrhosis and PBC, e.g NASH, liver fibrosis or PBC.

A method of modulating at least one integrin in a subject, the at least one integrin comprising an α_{V} subunit, the method comprising administering to the subject an effective amount of the pharmaceutical combination comprising administering a therapeutically effective amount of the pharmaceutical combination of the invention. In particular, the integrin being modulated is α_{V}β1.

The present invention provides a combination of two or more active ingredients that act on two or more distinct modes of NASH pathophysiology. A combination of an α_{V}β₁ integrin inhibitor, e.g. Compound 1, and SGLT1/2 inhibitor, e.g. licogliflozin, can address the metabolic, anti-inflammatory and antifibrotic pathways involved in NASH. The α_{V}β₁ integrin inhibitor Compound 1 and SGLT1/2 inhibitor licogliflozin impact distinct targets affecting different modes of NASH pathophysiology as evidenced by:
In freshly explanted fibrotic liver tissue obtained at the time of transplant from 5 patients with NASH, the α_{V}β₁ integrin inhibitor Compound 1, showed decreased expression of profibrotic genes, including COL1A1, which encodes the most abundant type of collagen produced in fibrosis, and TIMP1, which encodes the tissue inhibitor of metallopeptidase type 1 (TIMP-1). TIMP-1 is one of the three components of the Enhanced Liver Fibrosis (ELF) score, a noninvasive clinical diagnostic test to assess the likelihood of having clinically significant liver fibrosis.
Compound 1 showed potent and dose-dependent antifibrotic activity in animal models of NASH (CDAHFD) and liver fibrosis (CC14).
Without wishing to be bound by theory, it is believed from these findings that selective inhibition of integrin α_{V}β₁ by Compound 1 can provide anti-fibrotic benefits in NASH patients with advanced fibrosis.
PPAR (Peroxisome proliferator-activated receptor) modulators, such as seladelpar, elafibranor, lanifibranor, are suggested to treat PPAR-mediated conditions, including diabetes, cardiovascular diseases, Metabolic X syndrome, hypercholesterolemia, hypo-HDL-cholesterolemia, hyper-LDL-cholesterolemia, dyslipidemia, atherosclerosis, and obesity. PPAR agonists have been described to improve insulin sensitivity, glucose homeostasis, and lipid metabolism and reduces inflammation and showed effect in patients with NASH.
Lipid Modulator, such as Thyroid hormone receptor β (THRβ) agonist, are important modulators of lipid homeostasis, thermogenesis, and metabolic rate; e.g. resmetirom (MGL-3196) has shown statistically significant liver fat reduction and NASH resolution on biopsy.
Fibroblast growth factors (FGF) (i.e. FGF1, FGF19, and FGF21) have been identified as metabolic hormones; FGF21 analogs, such as pegbelfermin (BMS-986036), BMS-986171, efruxifermin; and FGF19 analogs, such as aldafermin, have showed in clinical studies an improvement of several NASH-related outcomes, including a decrease in liver fat content, plasma PRO-C3 levels, and plasma triglyceride levels.
Incretins, such as Glucagon-like peptide 1 (GLP-1) receptor agonists (GLP-1RAs) and dipeptidyl peptidase-4 (DPP4) inhibitors, e.g. GLP-1 agonists (e.g semaglutide) and DPP4) inhibitors (e.g. sitagliptin), approved for treatment of diabetes, have showed effects on NASH resolution without worsening of fibrosis.
Glucose pathway modulators, e.g. licogliflozin inhibits two closely related glucose cotransporters (SGLT1/2) in the gut and kidney.
The complementary effects of Compound 1 and of the additional therapeutic agents listed herein, for the treatments for fibrotic/cirrhotic diseases or disorders, e.g. liver diseases or disorders, can address the different aspects of these complex conditions, in patients in need of such treatment while demonstrating an acceptable safety and/or tolerability profile.
Compound 1 and licogliflozin are potent and highly specific for their respective targets.
The α_{V}β₁ integrin is not associated with changes in SGLT1 or SGLT2 expression or activity, and there is no known downstream intersection of the two pathways.
The anti-fibrotic effects of Compound 1 has not been described for licogliflozin.
The complementary effects of Compound 1 and licogliflozin can provide enhanced fibrosis reduction and/or improved clinical benefits in some patient populations.

Various embodiments of the invention are described herein. It will be recognized that features specified in each embodiment may be combined with other specified features to provide further embodiments of the present invention.

### BRIEF DESCRIPTION OF DRAWINGS

FIG. 1 is a graph showing that Compound 1 reduces expression of COL1A1 and TIMP1 in human cirrhotic NASH precision cut liver slices.

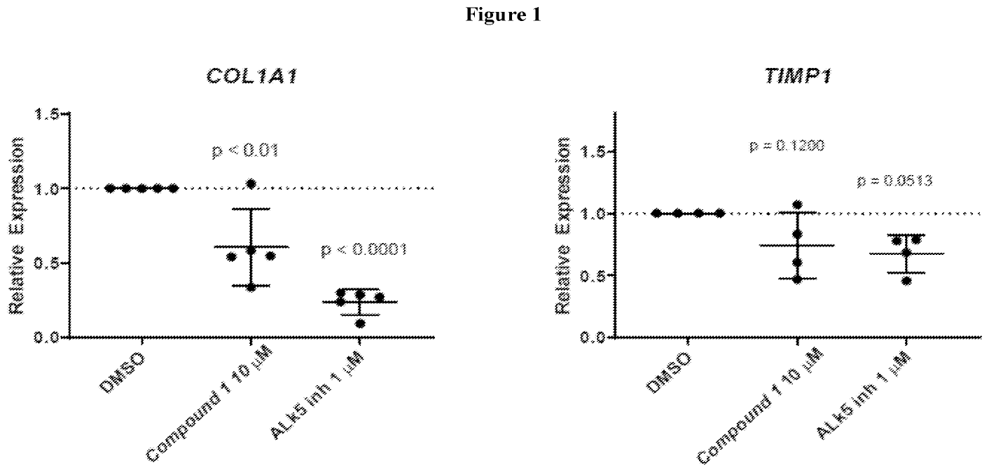

### DETAILED DESCRIPTION OF THE INVENTION

The present invention relates to a combination of two or more active ingredients with different Mechanisms of Action (MoA) that provide additional benefits for improving treatment efficacy and response rates.

The present disclosure relates to a pharmaceutical combination comprising, separate or together, at least an α_{V}β₁ integrin inhibitor and at least one additional therapeutic agent, for simultaneous, sequential, or separate administration. The invention further provides a medicament, comprising such a combination.

The present disclosure relates to a method of preventing, delaying or treating a liver disease or disorder, in a patient in need therefor, comprising administering a therapeutically effective amount of each active ingredient in the pharmaceutical combination. The pharmaceutical combination comprises (i) an α_{V}β₁ integrin inhibitor, e.g. Compound 1, and (ii) at least one additional therapeutic agent.

The present disclosure relates to a method of modulating at least one integrin in a subject, the at least one integrin comprising an α_{V} subunit, the method comprising administering to the subject an effective amount of the pharmaceutical combination comprising administering a therapeutically effective amount of the pharmaceutical combination of the invention. In particular, the integrin being modulated is α_{V}β1.

In another aspect, the invention provides a method for the treatment of a condition mediated by integrin, in particular a liver disease or an intestinal disease, in a subject in need thereof, comprising administering to said subject a pharmaceutical combination comprising:
1) an α_{V}β₁ integrin inhibitor, e.g. Compound 1, wherein the α_{V}β₁ integrin inhibitor is administered at a therapeutically effective dose, and
2) at least one additional therapeutic agent is selected from PPAR (Peroxisome proliferator-activated receptor) modulators, such as seladelpar, elafibranor, lanifibranor; Lipid Modulator, such as Thyroid hormone receptor β (THRβ) agonist, e.g. Resmetirom (MGL-3196) and VK-2809; FGF21 analogs, e.g. pegbelfermin (BMS-986036) and BMS-986171; FGF19 analogs, e.g. aldafermin; Incretins, such as Glucagon-like peptide 1 (GLP-1) receptor agonists (GLP-1RAs) (e.g. semaglutide) and dipeptidyl peptidase-4 (DPP4) inhibitors (e.g. sitagliptin).

In another aspect, the invention provides a method for the treatment of a condition mediated by integrin, in particular a liver disease or an intestinal disease, in a subject in need thereof, comprising administering to said subject a pharmaceutical combination comprising:
1) α_{V}β₁ integrin inhibitor, e.g. Compound 1, wherein α_{V}β₁ integrin inhibitor is administered at a therapeutically effective dose, and
2) an SGLT inhibitor, e.g. SGLT 1/2 inhibitor, e.g. licogliflozin.

The present invention provides a combination of two or more active ingredients that act on two or more distinct modes of NASH pathophysiology. A combination of an α_{V}β₁ integrin inhibitor, e.g. Compound 1, and at least one additional therapeutic agent as disclosed herein, has the potential to address the metabolic, anti-inflammatory and antifibrotic pathways involved in NASH. The α_{V}β₁ integrin inhibitor Compound 1 and at least one additional therapeutic agent as disclosed herein, impact distinct targets affecting different modes of NASH pathophysiology as evidenced by:
In freshly explanted fibrotic liver tissue obtained at the time of transplant from 5 patients with NASH, the α_{V}β₁ integrin inhibitor Compound 1, showed decreased expression of profibrotic genes, including COL1A1, which encodes the most abundant type of collagen produced in fibrosis, and TIMP1, which encodes the tissue inhibitor of metallopeptidase type 1 (TIMP-1). TIMP-1 is one of the three components of the Enhanced Liver Fibrosis (ELF) score, a noninvasive clinical diagnostic test to assess the likelihood of having clinically significant liver fibrosis.
Compound 1 showed potent and dose-dependent antifibrotic activity in animal models of NASH (CDAHFD) and liver fibrosis (CC14).
Without wishing to be bound by theory, it is believed from these findings that selective inhibition of integrin α_{V}β₁ by Compound 1 can provide anti-fibrotic benefits in NASH patients with advanced fibrosis.
PPAR (Peroxisome proliferator-activated receptor) modulators, such as seladelpar, elafibranor, lanifibranor, are suggested to treat PPAR-mediated conditions, including diabetes, cardiovascular diseases, Metabolic X syndrome, hypercholesterolemia, hypo-HDL-cholesterolemia, hyper-LDL-cholesterolemia, dyslipidemia, atherosclerosis, and obesity. PPAR agonists have been described to improve insulin sensitivity, glucose homeostasis, and lipid metabolism and reduces inflammation and showed effect in patients with NASH.
Lipid Modulator, such as Thyroid hormone receptor β (THRβ) agonist, are important modulators of lipid homeostasis, thermogenesis, and metabolic rate; e.g. resmetirom (MGL-3196) has shown statistically significant liver fat reduction and NASH resolution on biopsy.
Fibroblast growth factors (FGF) (i.e. FGF1, FGF19, and FGF21) have been identified as metabolic hormones; FGF21 analogs, such as pegbelfermin (BMS-986036), BMS-986171, efruxifermin; and FGF19 analogs, such as aldafermin, have showed in clinical studies an improvement of several NASH-related outcomes, including a decrease in liver fat content, plasma PRO-C3 levels, and plasma triglyceride levels.
Incretins, such as Glucagon-like peptide 1 (GLP-1) receptor agonists (GLP-1RAs) and dipeptidyl peptidase-4 (DPP4) inhibitors, e.g. GLP-1 agonists (e.g semaglutide) and DPP4) inhibitors (e.g. sitagliptin), approved for treatment of diabetes, have showed effects on NASH resolution without worsening of fibrosis.
Glucose pathway modulators, e.g. licogliflozin inhibits two closely related glucose cotransporters (SGLT1/2) in the gut and kidney.
Selective FXR agonists, such as obeticholic acid, have been shown to likely improve fibrosis in NASH and therefore may have a beneficial effect in delaying or even preventing cirrhosis.
The complementary effects of Compound 1 and of the additional therapeutic agents listed herein, for the treatments for fibrotic/cirrhotic diseases or disorders, e.g. liver diseases or disorders, can address the different aspects of these complex conditions, in patients in need of such treatment while demonstrating an acceptable safety and/or tolerability profile.
Compound 1 and licogliflozin are potent and highly specific for their respective targets.
The α_{V}β₁ integrin is not associated with changes in SGLT1 or SGLT2 expression or activity, and there is no known downstream intersection of the two pathways.
The anti-fibrotic effects of Compound 1 has not been described for licogliflozin.

The complementary effects of Compound 1 and licogliflozin can provide enhanced fibrosis reduction and/or improved clinical benefits in some patient populations.

### Embodiments (a)

1a. A pharmaceutical combination for simultaneous, sequentially or separate administration, comprising (i) an αVβ1 integrin inhibitor, e.g. Compound 1; and (ii) at least one additional therapeutic agent is selected from PPAR (Peroxisome proliferator-activated receptor) modulators, such as seladelpar, elafibranor, lanifibranor; Lipid Modulator, such as Thyroid hormone receptor β (THRβ) agonist, e.g. Resmetirom (MGL-3196) and VK-2809; FGF21 analogs, e.g. pegbelfermin, efruxifermin and BMS-986171; FGF19 analogs, e.g. aldafermin; Incretins, such as Glucagon-like peptide 1 (GLP-1) receptor agonists (GLP-1RAs) and dipeptidyl peptidase-4 (DPP4) inhibitors, e.g. GLP-1 agonists (e.g semaglutide), DPP4 inhibitors (e.g. sitagliptin), and FXR agonists (e.g. obeticholic acid).

2a. A pharmaceutical combination for simultaneous, sequentially or separate administration, comprising (i) an αVβ1 integrin inhibitor, e.g. Compound 1; and (ii) Thyroid hormone receptor β (THRβ) agonist, wherein the THRβ agonist is resmetirom.

3a. A pharmaceutical combination for simultaneous, sequentially or separate administration, comprising (i) an αVβ1 integrin inhibitor, e.g. Compound 1; and (ii) Thyroid hormone receptor β (THRβ) agonist, wherein the THRβ agonist is (2R,4S)-4-(3-chlorophenyl)-2-((4-(4-hydroxy-3-isopropylbenzyl)-3,5-dimethylphenoxy)methyl)-1,3,2-dioxaphosphinane 2-oxide.

4a. A pharmaceutical combination for simultaneous, sequentially or separate administration, comprising (i) an αVβ1 integrin inhibitor, e.g. Compound 1; and (ii) an FGF21 analog; preferably, wherein the FGF21 is pegbelfermin.

5a. A pharmaceutical combination for simultaneous, sequentially or separate administration, comprising (i) an αVβ1 integrin inhibitor, e.g. Compound 1; and (ii) an GLP-1 agonist, e.g semaglutide.

6a. A pharmaceutical combination for simultaneous, sequentially or separate administration, comprising (i) an α_{V}β₁ integrin inhibitor, e.g. Compound 1; and (ii) an SGLT inhibitor, e.g. SGLT 1/2 inhibitor.

7a. A pharmaceutical combination for simultaneous, sequential or separate administration, comprising (i) an α_{V}β₁ integrin inhibitor, e.g. Compound 1, wherein α_{V}β₁ integrin inhibitor is administered at a therapeutically effective dose; and (ii) an SGLT inhibitor, e.g. SGLT 1/2 inhibitor.

8a. A pharmaceutical combination for simultaneous, sequential or separate administration, comprising (i) an α_{V}β₁ integrin inhibitor, e.g. Compound 1, wherein α_{V}β₁ integrin inhibitor is administered at a therapeutically effective dose; and (ii) an FXR agonist (e.g. obeticholic acid).

9a. The pharmaceutical combination according to Embodiment 1a or 8a, wherein the α_{V}β₁ integrin inhibitor, e.g. Compound 1, is in a free form or a pharmaceutically acceptable salt, solvate, prodrug, ester and/or an amino acid conjugate thereof.

10a. The pharmaceutical combination according to any one of Embodiments 6a to 9a, wherein the SGLT inhibitor is selected from licogliflozin, dapagliflozin, canagliflozin, empagliflozin, ipragliflozin, ertugliflozin, mizagliflozin, sotagliflozin.

11a. The pharmaceutical combination according to Embodiment 10a, wherein the SGLT inhibitor is licogliflozin, in a free form, or as a pharmaceutically acceptable salt or in a crystalline form thereof.

12a. The pharmaceutical combination according to Embodiment 11a, comprising about 1 mg to about 300 mg of licogliflozin.

13a. The pharmaceutical combination according to Embodiment 12a, comprising from about 2 mg to about 200 mg of licogliflozin, from about 15 mg to about 150 mg, or from about 30 mg or about 150 mg of licogliflozin.

14a. The pharmaceutical combination according to Embodiment 12a, comprising about 1 mg, about 2 mg, about 15 mg, about 20 mg, about 30 mg, about 40 mg, about 50 mg, about 60 mg, about 70 mg, about 80 mg, about 90 mg, about 100 mg, or about 120 mg, about 150 mg, about 200 mg, about 250 mg, or about 300 mg of licogliflozin.

15a. The pharmaceutical combination according to Embodiment 12a, comprising about 15 mg to about 150 mg of licogliflozin.

16a. The pharmaceutical combination according to Embodiment 12a, comprising about 15 mg to about 75 mg of licogliflozin.

17a. The pharmaceutical combination according to Embodiment 12a, comprising about 15 mg to about 300 mg of licogliflozin.

18a. The pharmaceutical combination according to Embodiment 12a, comprising about 30 mg of licogliflozin.

19a. A pharmaceutical combination for simultaneous, sequential or separate administration, comprising: (i) Compound 1; and (ii) licogliflozin.

20a. A pharmaceutical combination for simultaneous, sequential or separate administration, comprising: (i) Compound 1; and (ii) from about 1 mg to about 300 mg of licogliflozin, e.g. from about 2 mg to about 200 mg of licogliflozin, or from about 15 mg to about 150 mg of licogliflozin.

21a. The pharmaceutical combination according to any one of Embodiments 10a to 20a, comprising an L-proline salt of licoglifozin.

22a. The pharmaceutical combination according to any one of Embodiments 1a to 21a, comprising a crystalline form of licogliflozin.

23a. The pharmaceutical combination according to Embodiment 20a, wherein said licogliflozin is an L-proline co-crystal of licogliflozin.

24a. The pharmaceutical combination according to any one of Embodiments 1a to 20a, comprising Compound 1 in a free form.

22a. The pharmaceutical combination according to any one of Embodiments 1a to 21a, comprising Compound 1 in a zwitterion form.

23a. The pharmaceutical combination according to any one of Embodiments 1a to 22a, wherein said combination is a fixed combination.

24a. The pharmaceutical combination according to any one of Embodiments 1a to 22a, wherein said combination is a free combination.

25a. A pharmaceutical combination according to any one of Embodiments 1a to 24a, for use in preventing, delaying or treating a condition mediated by integrin, in particular a liver disease or an intestinal disease.

26a. A method of preventing, delaying or treating a liver disease or disorder or an intestinal disease or disorder, in a subject in need thereof, comprising administering a therapeutically effective amount of the pharmaceutical combination according to any one of Embodiments 1a to 25a.

27a. The method according to Embodiment 26a, wherein the liver disease or disorder is a fibrotic or cirrhotic liver disease or disorder, selected from the group consisting of non-alcoholic fatty liver disease (NAFLD), non-alcoholic steatohepatitis (NASH), liver cirrhosis, alcohol-induced cirrhosis, cystic fibrosis-associated liver disease (CFLD), liver fibrosis, and progressive fibrosis of the liver caused by any of the diseases above or by infectious hepatitis.

28a. The method according to Embodiment 26a, wherein the liver disease or disorder is non-alcoholic fatty liver disease (NAFLD), non-alcoholic steatohepatitis (NASH), primary biliary cirrhosis (PBC), liver fibrosis, or liver cirrhosis.

29a. The method according to Embodiment 26a, wherein the liver disease or disorder is non-alcoholic fatty liver disease, (NAFLD).

30a. The method according to Embodiment 26a, wherein the liver disease or disorder is non-alcoholic steatohepatitis (NASH).

31a. The method according to Embodiment 30a, further comprising resolution of steatohepatitis.

32a. The method according to Embodiment 26a, wherein the liver disease or disorder is liver fibrosis.

33a. The method according to any one of Embodiments 30a to 32a, further comprising improvement in liver fibrosis.

34a. The method according to any one of Embodiments 30a to 33a, further comprising improvement in liver cirrhosis.

35a. The method according to any one of Embodiments 26a to 34a, wherein the SGLT inhibitor is administered in the evening.

36a. The method according to Embodiment 35a, thereby reducing the risk of diarrhea associated with the administration of the SGLT inhibitor.

### Definitions

For purposes of interpreting this specification, the following definitions will apply and whenever appropriate, terms used in the singular will also include the plural and vice versa.

As used herein, the term “a”, “an” or the like refers to one or more.

As used herein, the term “about” in relation to a numerical value x means +/−10%, unless the context dictates otherwise.

As used herein, the term “FXR agonist” refers to an agent that directly binds to and upregulates the activity of FXR which may be referred to as bile acid receptor (BAR) or NR1H4 (nuclear receptor subfamily 1, group H, member 4) receptor. FXR agonists may act as agonists or partial agonists of FXR. The agent may be e.g. a small molecule, an antibody or a protein, preferably a small molecule. The activity of a FXR agonist may be measured by several different methods, e.g. in an in vitro assay using the fluorescence resonance energy transfer (FRET) cell free assay as described in Pellicciari, et al. Journal of Medicinal Chemistry, 2002 vol. 15, No. 45:3569-72.

As used herein, the terms “salt” or “salts” refers to an acid addition or base addition salt of a compound of the invention. “Salts” include in particular “pharmaceutical acceptable salts.”

As used herein, the term “amino acid conjugate” refers to conjugates of compounds with any suitable amino acid. Preferably, such suitable amino acid conjugates of a compound will have the added advantage of enhanced integrity in bile or intestinal fluids. Suitable amino acids include but are not limited to glycine, taurine and acyl glucuronide. Thus, the present invention encompasses, for example, the glycine, taurine and acyl glucuronide conjugates of the FXR agonist (e.g. nidufexor and obeticholic acid) or α_{V}β₁ integrin inhibitor (e.g. Compound 1).

As used herein, the term “pharmaceutically acceptable” means a nontoxic material that does not interfere with the effectiveness of the biological activity of the active ingredient(s).

As used herein the term “prodrug” refers to compound that is converted in vivo to the compounds of the present invention. A prodrug is active or inactive. It is modified chemically through in vivo physiological action, such as hydrolysis, metabolism and the like, into a compound of this invention following administration of the prodrug to a subject. The suitability and techniques involved in making and using pro-drugs are well known by those skilled in the art. Suitable prodrugs are often pharmaceutically acceptable ester derivatives.

As used herein, the terms “patient” or “subject” are used interchangeably and refer to a human.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition, such as e.g. liver cirrhosis; or to preventing or delaying a need for liver transplantation. For example, treating NASH using for example, any of the combinations disclosed herein, may refer to ameliorating, alleviating or modulating at least one of the symptoms or pathological features associated with NASH; e.g. hepatosteatosis, hepatocellular ballooning, hepatic inflammation and fibrosis; e.g. may refer to slowing progression, reducing or stopping at least one of the symptoms or pathological features associated with NASH, e.g. hepatosteatosis, hepatocellular ballooning, hepatic inflammation and fibrosis. It may also refer to preventing or delaying liver cirrhosis or a need for liver transplantation.

As used herein, the term “therapeutically effective amount” refers to an amount of the integrin inhibitor and/or the at least one additional therapeutic agent of the pharmaceutical combination of the invention, individually or in combination, e.g. the α_{V}β₁ integrin inhibitor and/or the at least one additional therapeutic agent, which is sufficient to achieve the respective stated effect. Accordingly, a therapeutically effective amount of the α_{V}β₁ integrin inhibitor and/or the at least one additional therapeutic agent, e.g. Compound 1 and/or an FXR agonist, used for the treatment or prevention of a liver disease or disorder as hereinabove defined is an amount sufficient for the treatment or prevention of such a disease or disorder individually or in combination.

By “therapeutic regimen” is meant the pattern of treatment of an illness, e.g., the pattern of dosing used during the treatment of the disease or disorder.

As used herein, a subject is “in need of” a treatment if such subject would benefit biologically, medically or in quality of life from such treatment.

As used herein, the term “liver disease or disorder” encompasses one, a plurality, or all of non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis and liver fibrosis.

As used herein, the term NAFLD may encompass the different stages of the disease: hepatosteatosis, NASH, fibrosis and cirrhosis.

As used herein, the term NASH may encompass steatosis, hepatocellular ballooning and lobular inflammation.

As herein defined, “combination” refers to either a fixed combination in one unit dosage form (e.g., capsule, tablet, or sachet), free (i.e. non-fixed) combination, or a kit of parts for the combined administration where an α_{V}β₁ integrin inhibitor of the present invention and one or more “combination partner” (i.e. the at least one additional therapeutic agent, such as e.g. a non-bile acid derived farnesoid X receptor (FXR) agonist or a pharmaceutically acceptable salt or solvate thereof, also referred to as or “co-agent”) may be administered independently at the same time or separately within time intervals, especially where these time intervals allow that the combination partners show a cooperative, e.g. synergistic effect.

The terms “co-administration” or “combined administration” or the like as utilized herein are meant to encompass administration of the at least one additional therapeutic agent to a single subject in need thereof (e.g. a patient), and the at least one additional therapeutic agent are intended to include treatment regimens in which the α_{V}β₁ integrin inhibitor and the at least one additional therapeutic agent such as the FXR agonist are not necessarily administered by the same route of administration and/or at the same time. Each of the components of the combination of the present invention may be administered simultaneously or sequentially and in any order. Co-administration comprises simultaneous, sequential, overlapping, interval, continuous administrations and any combination thereof.

The term “pharmaceutical combination” as used herein means a pharmaceutical composition that results from the combining (e.g. mixing) of more than one active ingredient and includes both fixed and free combinations of the active ingredients.

The term “fixed combination” means that the active ingredients, i.e. 1) an α_{V}β₁ integrin inhibitor, e.g. Compound 1 (as defined herein), and 2) the at least one additional therapeutic agent, e.g. a non-bile acid derived FXR agonist, e.g. nidufexor, are both administered to a patient simultaneously in the form of a single entity or dosage.

The term “free combination” means that the active ingredients as herein defined are both administered to a patient as separate entities either simultaneously, concurrently or sequentially with no specific time limits, and in any order, wherein such administration provides therapeutically effective levels of the two compounds in the body of the patient.

By “simultaneous administration”, it is meant that 1) an α_{V}β₁ integrin inhibitor, e.g. Compound 1 (as defined herein), and 2) the at least one additional therapeutic agent, e.g. the FXR agonist, e.g. nidufexor, are administered on the same day. The two active ingredients can be administered at the same time (for fixed or free combinations) or one at a time (for free combinations).

According to the invention, “sequential administration”, may mean that during a period of two or more days of continuous co-administration only one of 1) an α_{V}β₁ integrin inhibitor, e.g. Compound 1 (as defined herein), and 2) the at least one additional therapeutic agent, e.g. the FXR agonist, e.g. nidufexor, is administered on any given day.

By “overlapping administration”, it is meant that during a period of two or more days of continuous co-administration, there is at least one day of simultaneous administration and at least one day when only one of 1) an α_{V}β₁ integrin inhibitor, e.g. Compound 1 (as defined herein), and 2) the at least one additional therapeutic agent, e.g. the FXR agonist, is administered.

By “continuous administration”, it is meant a period of co-administration without any void day. The continuous administration may be simultaneous, sequential, or overlapping, as described above.

The term “Compound 1” means (S)-2-(4-methyltetrahydro-2H-pyran-4-carboxamido)-9-(5,6,7,8-tetrahydro-1,8-naphthyridin-2-yl)nonanoic acid (shown below). The term includes a stereoisomer, an enantiomer, in free form, a zwitterion, a polymorph, a pharmaceutically acceptable salt, a solvate, a hydrate, a prodrug, an ester, or an amino acid conjugate thereof; and is also intended to represent unlabeled forms as well as isotopically labeled forms of the compound.

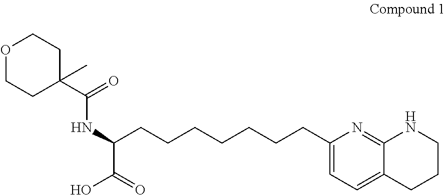 [SMILES: CC1(C(=O)N[C@@H](CCCCCCCc2ccc3c(n2)NCCC3)C(=O)O)CCOCC1]

The term “licogliflozin” means ((2S,3R,4R,5S,6R)-2-(3-((2,3-dihydrobenzo[b][1,4]dioxin-6-yl)methyl)-4-ethylphenyl)-6-(hydroxymethyl)tetrahydro-2H-pyran-3,4,5-triol (shown below). The term includes a stereoisomer, an enantiomer, in free form, a zwitterion, a polymorph, a pharmaceutically acceptable salt, a solvate, a hydrate, a prodrug, an ester, or an amino acid conjugate thereof; and is also intended to represent unlabeled forms as well as isotopically labeled forms of the compound.

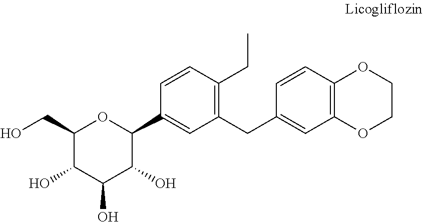 [SMILES: CCc1ccc([C@@H]2O[C@H](CO)[C@@H](O)[C@H](O)[C@H]2O)cc1Cc1ccc2c(c1)OCCO2]

Any named compound includes a stereoisomer, an enantiomer, in free form, a zwitterion, a polymorph, a pharmaceutically acceptable salt, a solvate, a hydrate, a prodrug, an ester, or an amino acid conjugate thereof; and is also intended to represent unlabeled forms as well as isotopically labeled forms of the compound.

Unless otherwise specified, the amount of Compound 1 or the additional therapeutic agent refers to the amount of each in a free form.

α_{V}β₁ Integrin Inhibitor

According to an embodiment of the invention, the α_{V}β₁ integrin inhibitor is Compound 1. As defined above, the term “Compound 1” also includes a stereoisomer, an enantiomer, in free form (including a zwitterion), a polymorph, a pharmaceutically acceptable salt, a solvate, a hydrate, a prodrug, an ester, or an amino acid conjugate thereof, e.g. HCl or TFA salt.

In one embodiment, the amino acid conjugate is a glycine conjugate, taurine conjugate or acyl glucuronide conjugate.

In one embodiment, Compound 1 is also intended to represent unlabeled forms as well as isotopically labeled forms of the compound.

### Additional Therapeutic Agents or Combination Partners

The terms “additional therapeutic agent” and “combination partner” are herein used interchangeably. A combination of an α_{V}β₁ integrin inhibitor with the combination partner can address the metabolic, anti-inflammatory and anti-fibrotic pathways involved in NASH. According to an embodiment of the invention, at least one therapeutic agent may be beneficially combined with the disclosed α_{V}β₁ integrin inhibitor (e.g., Compound 1) in the treatment or prevention of a liver disease or disorder or an intestinal disease or disorder in a subject in need thereof.

The at least one additional therapeutic agent is at least one of the following: FXR agonist (M480 (Metacrine), NTX-023-1 (Ardelyx), INV-33 (Innovimmune)), Steroyl-CoA desaturase-1 (SCD-1) inhibitor (e.g., arachidyl amido cholanoic acid (Aramchol™)), THR-β agonist (e.g., MGL-3196 (Resmetirom), VK-2809, MGL-3745 (Madrigal)), galectin-2 inhibitor (e.g., GR-MD-02/Belapectin), PPAR agonist (e.g., saroglitazar, seladelpar, elafibranor, lanifibranor, lobeglitazone, pioglitazone, IVA337 (Inventiva), CER-002 (Cerenis), MBX-8025 (Seladelpar)), GLP-1 agonist (e.g., exenatide, liraglutide, semaglutide, NC-101 (Naia Metabolic), G-49 (Astrazeneca), ZP2929 (BI/Zealand), PB-718 (Peg Bio)), FGF agonist (e.g., pegbelfermin (ARX618), BMS-986171, NGM-282, NGM-313, YH25724, and proteins disclosed in WO2013049247, WO2017021893 and WO2018146594), tirzepatide, pyruvate synthase inhibitors (e.g., nitazoxanide), Apoptosis signal-regulating kinase 1 (ASK1) inhibitor (e.g., selonsertib (GS-4997), GS-444217), Acetyl-CoA carboxylase (ACC) inhibitor (e.g., firsocostat (GS-0976), PF-05221304, gemcabene (Gemphire)), CCR inhibitor (e.g., AD-114 (AdAlta), Bertilimumab (Immune), CM-101 (ChemomAb), CCX-872 (ChemoCentryx), Cenicriviroc), thiazolidinedione (e.g, MSDC-0602K, Pioglitazone), sodium-glucose co-transporter-2 and 1 (SGLT1/2) inhibitor (e.g., Remogliflozin, luseogliflozin, dapagliflozin, licogliflozin), DPP-4 inhibitor (sitagliptin, saxagliptin, vildagliptin, linagliptin, evogliptin, gemigliptin, anagliptin, teneligliptin, alogliptin, trelagliptin, omarigliptin, gosogliptin, dutogliption), insulin receptor agonist (e.g. ORMD 0801 (Oramed)), SGLT-2 inhibitor with DPPP inhibitor (e.g. empagliflozin and linagliptin), insulin sensitizer (e.g., MSDC-0602K (Octeta/Cirius)), CCR2/5 inhibitor (e.g., CVC (Allergan), anti-BMP9 antibodies (e.g., the antibodies described in WO2016193872); a compound selected from the group consisting of ((R)-3-amino-4-(5-(4-((5-chloropyridin-2-yl)oxy)phenyl)-2H-tetrazol-2-yl)butanoic acid; (R)-3-amino-4-(5-(4-((5-chloro-3-fluoropyridin-2-yl)oxy)phenyl)-2H-tetrazol-2-yl)butanoic acid; (R)-3-amino-4-(5-(3-((5-(trifluoro-methyl)pyridin-2-yl)oxy)phenyl)-2H-tetrazol-2-yl)butanoic acid; (R)-3-amino-4-(5-(4-((5-(trifluoro-methyl)pyridin-2-yl)oxy)phenyl)-2H-tetrazol-2-yl)butanoic acid; (S)-3-amino-4-(5-(4-((5-chloro-3-fluoropyridin-2-yl)oxy)phenyl)-2H-tetrazol-2-yl)butanoic acid; (R)-3-amino-4-(5-(4-phenethoxyphenyl)-2H-tetrazol-2-yl)butanoic acid; and (R)-3-amino-4-(5-(4-(4-chlorophenoxy)-phenyl)-2H-tetrazol-2-yl)butanoic acid; or a pharmaceutically acceptable salt thereof, or any combination thereof.

The FXR agonist as used herein refers, for example, to compounds disclosed in: WO2016/096116, WO2016/127924, WO2017/218337, WO2018/024224, WO2018/075207, WO2018/133730, WO2018/190643, WO2018/214959, WO2016/096115, WO2017/118294, WO2017/218397, WO2018/059314, WO2018/085148, WO2019/007418, CN109053751, CN104513213, WO2017/128896, WO2017/189652, WO2017/189663, WO2017/189651, WO2017/201150, WO2017/201152, WO2017/201155, WO2018/067704, WO2018/081285, WO2018/039384, WO2015/138986, WO2017/078928, WO2016/081918, WO2016/103037, and WO2017/143134.

The FXR agonist is preferably selected from: nidufexor, obeticholic acid (6α-ethyl-chenodeoxycholic acid), cilofexor (GS-9674, Px-102), INT-767, AKN-083, TERN-101 (LY2562175):

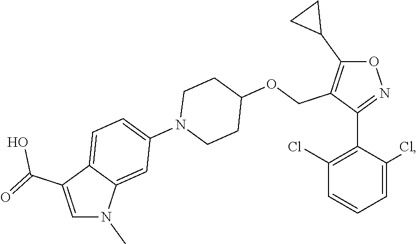 [SMILES: Cn1cc(C(=O)O)c2ccc(N3CCC(OCc4c(-c5c(Cl)cccc5Cl)noc4C4CC4)CC3)cc21]

### EYP001 (PXL007):

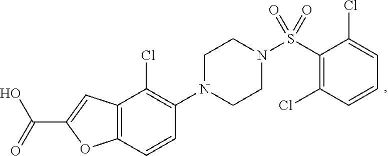 [SMILES: O=C(O)c1cc2c(Cl)c(N3CCN(S(=O)(=O)c4c(Cl)cccc4Cl)CC3)ccc2o1]

### EDP-305:

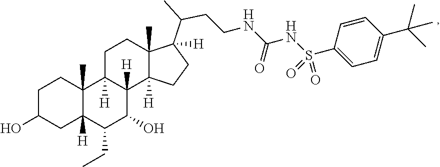

and 4-((N-benzyl-8-chloro-1-methyl-1,4-dihydrochromeno[4,3-c]pyrazole-3 carboxamido)methyl)benzoic acid, M480 (Metacrine), a pharmaceutically acceptable salt, prodrug and/or ester thereof and/or an amino acid conjugate thereof, e.g. meglumine salt. In some embodiments, the FXR agonist is other than tropifexor. In some embodiments, the FXR agonist is a non-bile acid derived FXR agonist.

According to an embodiment of the invention, the at least one additional therapeutic agent is a non-bile acid derived FXR agonist, e.g. nidufexor. As defined above, the term “nidufexor” also includes a stereoisomer, an enantiomer, in free form, a zwitterion, a polymorph, a pharmaceutically acceptable salt, a solvate, a hydrate, a prodrug, an ester, or an amino acid conjugate thereof.

According to an embodiment of the invention, the at least one additional therapeutic agent is a SGLT1/2 inhibitor, e.g. licogliflozin ((2S,3R,4R,5S,6R)-2-(3-((2,3-dihydrobenzo[b][1,4]dioxin-6-yl)methyl)-4-ethylphenyl)-6-(hydroxymethyl)tetrahydro-2H-pyran-3,4,5-triol, as shown below).

Licogliflozin (also known as LIK066) has the following chemical structure:

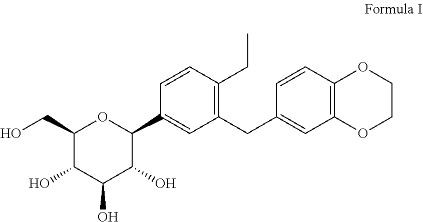 [SMILES: CCc1ccc([C@@H]2O[C@H](CO)[C@@H](O)[C@H](O)[C@H]2O)cc1Cc1ccc2c(c1)OCCO2]

Licogliflozin of Formula I may be in a free form, a pharmaceutically acceptable salt form or complex form. An example of a pharmaceutical acceptable complex is a proline complex, such as di-L-proline of Formula I(a) and di-S-proline (formula not shown):

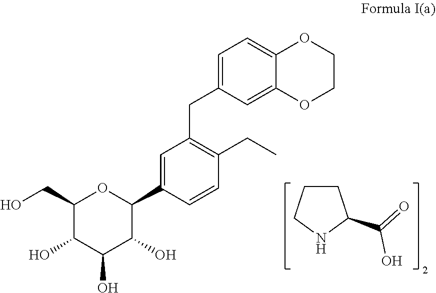

Licogliflozin is a potent inhibitor of the sodium glucose co-transporters (SGLTs) 1 and 2 that decreases absorption of glucose in the gut and reabsorption in the kidney. Licogliflozin was found to be safe and tolerated, had a favorable pharmacokinetic profile, and resulted in up to 3% placebo-adjusted weight loss over just 2 weeks in both healthy subjects and patients with T2DM. Licogliflozin at 150 mg daily dose results in a significant weight loss in obese patients (˜6%) after a 12 week treatment. Furthermore, a twelve week treatment with licogliflozin at 150 mg once daily, in normoglycemic and dysglycemic subjects was generally safe and well tolerated with diarrhea observed as a dose-limiting toxicity.

As described above, licogliflozin of Formula I includes a pharmaceutically acceptable salt or complex form. The latter includes a licogliflozin proline complex, such as licogliflozin di-L-proline complex of Formula I(a) and licogliflozin di-S-proline complex.

According to an embodiment of the invention, the at least one additional therapeutic agent is an PPAR (Peroxisome proliferator-activated receptor) modulators, such as seladelpar, elafibranor and lanifibranor.

According to an embodiment of the invention, the at least one additional therapeutic agent is a lipid modulator, such as thyroid hormone receptor β (THRβ) agonist, e.g. Resmetirom (MGL-3196) and VK-2809. VK-2809 refers to (2R,4S)-4-(3-chlorophenyl)-2-((4-(4-hydroxy-3-isopropylbenzyl)-3,5-dimethylphenoxy)methyl)-1,3,2-dioxaphosphinane 2-oxide (shown below)

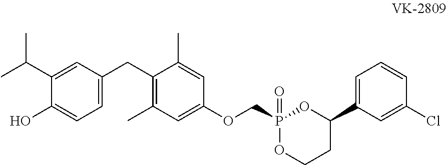 [SMILES: Cc1cc(OC[P@]2(=O)OCC[C@H](c3cccc(Cl)c3)O2)cc(C)c1Cc1ccc(O)c(C(C)C)c1]

According to an embodiment of the invention, the at least one additional therapeutic agent is an FGF21 analog, e.g. pegbelfermin (BMS-986036), efruxifermin and BMS-986171.

According to an embodiment of the invention, the at least one additional therapeutic agent is an FGF19 analog, e.g. aldafermin.

According to an embodiment of the invention, the at least one additional therapeutic agent is an incretin, such as Glucagon-like peptide 1 (GLP-1) receptor agonists (GLP-1RAs) (e.g. semaglutide) and dipeptidyl peptidase-4 (DPP4) inhibitors (e.g. sitagliptin).

In one embodiment, the amino acid conjugate is a glycine conjugate, taurine conjugate or acyl glucuronide conjugate.

### Pharmaceutical Compositions

The α_{V}β₁ integrin inhibitor or the at least one additional therapeutic agent each may be used as a pharmaceutical composition with a pharmaceutically acceptable carrier. For example, such a composition may contain, in addition to the α_{V}β₁ integrin inhibitor or an FXR agonist, carriers, various diluents, fillers, salts, buffers, stabilizers, solubilizers, and other materials known in the art. The characteristics of the carrier will depend on the route of administration.

The pharmaceutical composition for use in the disclosed methods may be a free combination of a pharmaceutical composition containing an α_{V}β₁ integrin inhibitor (e.g. Compound 1), and a pharmaceutical composition containing any of the additional therapeutic agents discussed above (e.g. a SGLT1/2 inhibitor, e.g. licogliflozin), each as described above.

The pharmaceutical composition for use in the disclosed methods may also be a combination pharmaceutical composition in a single dosage unit that contains the α_{V}β₁ integrin inhibitor and the at least one additional therapeutic agent for treatment of the particular targeted disorder. For example, a pharmaceutical composition includes the α_{V}β₁ integrin inhibitor (e.g. Compound 1) and any of the additional therapeutic agents disclosed above (e.g. a SGLT1/2 inhibitor, e.g. licogliflozin) discussed above in the treatment or prevention of liver disease or disorder or an intestinal disease or disorder. Such additional therapeutic agents are included in the combination pharmaceutical composition to produce a synergistic effect with the α_{V}β₁ integrin inhibitor.

### Modes of Administration

The pharmaceutical composition of the invention can be formulated to be compatible with its intended route of administration (e.g. oral compositions generally include an inert diluent or an edible carrier). Other non-limiting examples of routes of administration include parenteral (e.g. intravenous), intradermal, subcutaneous, oral (e.g. inhalation), transdermal (topical), transmucosal, and rectal administration.

### Diseases

As hereinabove defined, the fibrotic or cirrhotic disease or disorder can be a liver disease or disorder, or renal fibrosis.

In one embodiment of the invention, the pharmaceutical combination (as herein defined) is for the treatment or prevention of a fibrotic disease or disorder, e.g. a liver disease or disorder, e.g. a chronic liver disease, e.g. a liver disease or disorder selected from the group consisting of PBC, NAFLD, NASH, drug-induced bile duct injury, gallstones, liver cirrhosis, alcohol-induced cirrhosis, cystic fibrosis-associated liver disease (CFLD), bile duct obstruction, cholelithiasis, liver fibrosis. In one embodiment of the invention, the pharmaceutical combination (as herein defined) is for the treatment or prevention of fibrosis, e.g. renal fibrosis or liver fibrosis.

According to one embodiment of the invention, the liver diseases or disorders refer to NAFLD, e.g. any stages of NAFLD, e.g. any of steatosis, NASH, fibrosis and cirrhosis.

In one embodiment of the invention, there is provided a pharmaceutical combination of the invention for the improvement of liver fibrosis without worsening of steatohepatitis.

In another embodiment of the invention, there is provided a pharmaceutical combination of the invention for obtaining a complete resolution of steatohepatitis without worsening, e.g. improving, of liver fibrosis.

In another embodiment of the invention, there is provided a pharmaceutical combination of the invention for preventing or treating steatohepatitis and liver fibrosis.

In yet another embodiment of the invention, there is provided a pharmaceutical combination of the invention for reducing at least one of the features of the NAS score, i.e. one of hepatosteatosis, hepatic inflammation and hepatocellular ballooning; e.g. at least two features of the NAS score, e.g. hepatosteatosis and hepatic inflammation, or hepatosteatosis and hepatocellular ballooning, or hepatocellular ballooning and hepatic inflammation.

In a further embodiment of the invention, there is provided a pharmaceutical combination of the invention for reducing at least one or two features of the NAS score and liver fibrosis, e.g. for reducing hepatic inflammation and liver fibrosis, or hepatosteatosis and liver fibrosis or hepatocellular ballooning and liver fibrosis.

In yet a further embodiment of the invention there is provided a pharmaceutical combination for treating or preventing, stage 3 fibrosis to stage 1 fibrosis, e.g. stage 3 and/or stage 2 and/or stage 1 fibrosis.

In one embodiment of the invention, the pharmaceutical combination (as herein defined) is for the treatment or prevention of an intestinal disease or disorder.

### Patients/Subjects

According to the invention, the subjects receiving the pharmaceutical combination of the invention can be affected or at risk of a fibrotic disease or disorder, e.g. a liver disease or disorder, as hereinabove defined.

In some embodiments of the invention, the subject is obese or overweight.

In other embodiments of the invention, the subject may be a diabetic subject, e.g. may have type 2 diabetes. The subject may have high blood pressure and/or high blood cholesterol level.

### Dosing Regimens

Depending on the compound used, the targeted disease or disorder and the stage of such disease or disorder, the dosing regimen, i.e. administered doses and/or frequency, may vary.

The dosing frequency will depend on; inter alia, the phase of the treatment regimen.

According to the invention, licogliflozin (as hereinabove defined) is administered at a dose of e.g. about 20 mg, e.g. about 30 mg, e.g. about 50 mg, e.g. about 60 mg, e.g. about 80 mg, e.g. about 90 mg, e.g. about 100 mg, e.g. about 120 mg, e.g. about 150 mg. Such doses may be for oral administration licogliflozin.

In some aspects, licogliflozin is administered at a dose of about 30 mg.

### Kits for Treatment of Liver Fibrotic Disease or Disorder

Accordingly, there are provided a pharmaceutical kit comprising: a) an α_{V}β₁ integrin inhibitor, e.g. Compound 1; b) the at least one additional therapeutic agent, e.g. a FXR agonist, e.g. non-bile acid derived FXR agonists, e.g. nidufexor; a SGLT1/2 inhibitor, e.g. licogliflozin, or any of the additional therapeutic agents described above, and c) means for administering the α_{V}β₁ integrin inhibitor and the at least one additional therapeutic agent, to a subject affected by a liver disease or disorder; and optionally d) instructions for use.

In one embodiment of the invention, there is provided a combination package comprising: a) an α_{V}β₁ integrin inhibitor, e.g. Compound 1; and b) at least one individual dose of at least one additional therapeutic agent as hereinabove defined, e.g. at least one individual dose of a FXR agonist, e.g. non-bile acid derived FXR agonists, e.g. nidufexor, of a SGLT1/2 inhibitor, e.g. licogliflozin, or of any of the additional therapeutic agents described above. The combination package may further comprise instructions for use.

### EXAMPLES

The examples and embodiments described herein are for illustrative purposes only and various modifications or changes in light thereof will be suggested to persons skilled in the art and are to be included within the spirit and purview of this application and scope of the appended claims. All publications, patents, and patent applications cited herein are hereby incorporated by reference for all purposes.

### Example 1—Synthesis

(S)-2-(4-methyltetrahydro-2H-pyran-4-carboxamido)-9-(5,6,7,8-tetrahydro-1,8-naphthyridin-2-yl)nonanoic acid (Compound 1) may be prepared according to Scheme A below.

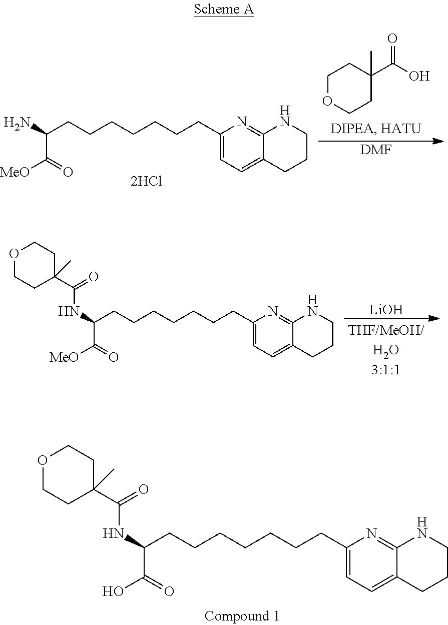

Step 1:

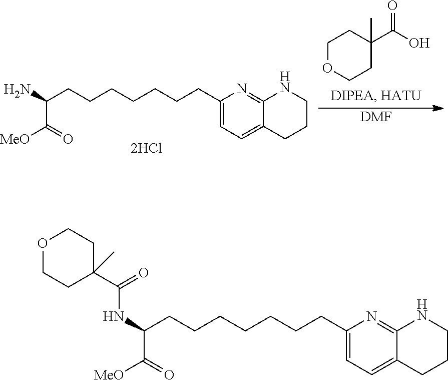

To a solution of methyl (S)-2-amino-9-(5,6,7,8-tetrahydro-1,8-naphthyridin-2-yl)nonanoate in DMF was added DIPEA (10 equiv) followed by 4-methyltetrahydro-2H-pyran-4-carboxylic acid (1.1 equiv) and HATU (1.1 equiv). The reaction was allowed to stir at room temperature while monitoring reaction progress by LCMS. When the starting material had been consumed, the reaction was diluted with 1 N NaOH and extracted with EA, washed with brine, dried over sodium sulfate, and concentrated. The crude residue was purified by silica gel chromatography to afford the depicted compound.

Step 2:

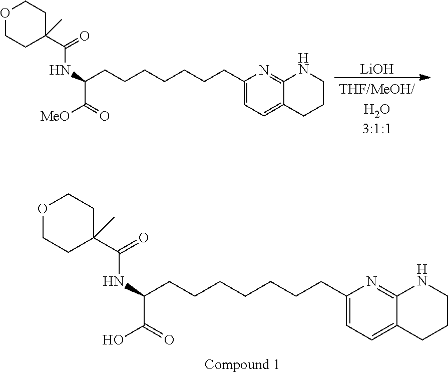

To a solution of the depicted ester in an appropriate solvent mixture such as THF/MeOH/H₂O or THF/EtOH/H₂O was added LiOH (3-5 equiv). The reaction was allowed to stir at room temperature while monitoring reaction progress by LCMS. Upon completion, the reaction was concentrated and purified by reverse phase preparative HPLC to afford the Compound 1 as the TFA salt. LCMS theoretical m/z=432.2 [M+H]+. found 432.3.

### Example 2—Solid Phase Integrin α_{V}β₁ or α_{V}β₆ Binding Assay

Microplates were coated with recombinant human integrin α_{V}β₁ or α_{V}β₆ (2 μg/mL) in PBS (100 μL/well 25° C., overnight). The coating solution was removed, washed with wash buffer (0.05% Tween 20; 0.5 mM MnCl₂; in 1×TBS). The plate was blocked with 200 μL/well of Block Buffer (1% BSA; 5% sucrose; 0.5 mM MnCl₂; in 1×TBS) at 37° C. for 2 h. Dilutions of Compound 1 and recombinant TGFβ1 LAP (0.67 μg/mL) in binding buffer (0.05% BSA; 2.5% sucrose; 0.5 mM MnCl₂; in 1×TBS) were added. The plate was incubated for 2 hours at 25° C., washed, and incubated for 1 hour with Biotin-Anti-hLAP. Bound antibody was detected by peroxidase-conjugated streptavidin. The IC₅₀ values for the testing compound were calculated by a four-parameter logistic regression.

### Example 3—Proximity-Based Integrin α_{V}β₁ or α_{V}β₆ Binding Assay

Compound 1 was tested for α_{V}β₁ or α_{V}β₆ integrin biochemical potency using the ALPHASCREEN® (Perkin Elmer, Waltham, Mass.) proximity-based assay (a bead-based, non-radioactive Amplified Luminescent Proximity Homogeneous Assay) as described previously (Ullman E F et al., Luminescent oxygen channeling immunoassay: Measurement of particle binding kinetics by chemiluminescence. Proc. Natl. Acad. Sci. USA, Vol. 91, pp. 5426-5430, June 1994). To gauge the potency of inhibitors of binding to human integrin α_{V}β₁ or αᵥβ₆, the inhibitor compound and the integrin were incubated together with recombinant TGFβ₁ LAP and biotinylated anti-LAP antibody plus acceptor and donor beads, following the manufacturer's recommendations. The donor beads were coated with streptavidin. The acceptor beads had a nitrilotriacetic acid Ni chelator, for binding to a 6×His-tag on human integrin α_{V}β₁ or αᵥβ₆. All incubations occurred at room temperatures in 50 mM Tris-HCl, pH 7.5, 0.1% BSA supplemented with 1 mM each CaCl₂ and MgCl₂. The order of reagent addition was as follows: 1. α_{V}β₁ or α_{V}β₆ integrin, test inhibitor Compound 1, LAP, biotinylated anti-LAP antibody and acceptor beads were all added together. 2. After 2 hours, donor beads were added. After another 30 min incubation, samples were read.

### Example 4—Results of α_{V}β₁ or α_{V}β₆ Integrin Inhibition

The IC₅₀ values obtained for α_{V}β₁ and α_{V}β₆ integrin inhibition for Compound 1 obtained in Examples 2 and 3 are in Table 1 below:

TABLE 1

| | | |
|---|---|---|
| Solid Phase Assay | αvβ₁ | <50 |
| (IC₅₀ in nM) | αvβ₆ | <50 |
| Proximity-Based Assy | αvβ₁ | 50 to below 250 |
| (IC₅₀ in nM) | αvβ₆ | <50 |

### Example 5—In Vivo Efficacy Study 1

Adult male C57BL/6J mice are housed with ad libitum access to water and food. Mice are fed a HF/NASH diet (40 kcal % fat, 2% cholesterol, 40 kcal % carbohydrate, Research Diets, D09100301 or S Sniff Special Diets, supplemented with a fructose-sucrose solution (42 g/L, 55% fructose and 45% sucrose by weight) in drinking water). Age-matched animals are maintained on regular chow (Normal Diet, ND, Kliba Nafag, 3892) and received tap water. Mice are subjected to HF/NASH diet for a total of 20 weeks.

At week 8 of HF/NASH feeding, HF/NASH animals are randomized to treated and untreated groups according to body weight, total lean and fat masses, and liver fat measured by MRI. The study comprises four groups of mice: Group 1: Normal Diet/Water (n=7); Group 2: HF/NASH+nidufexor (n=9); Group 3: HF/NASH+Compound 1 (n=9); and Group 4: HF/NASH+nidufexor+Compound 1 (n=9). Body weight is measured weekly. Fat and lean masses are measured at 0, 4, 7, 14 and 20 weeks of HF/NASH feeding using a mouse body composition nuclear magnetic resonance (NMR) analyzer; and liver fat is assessed at 8, 12, 16 and 20 weeks of HF/NASH feeding using magnetic resonance imaging (MRI).

### Example 6—In Vivo Efficacy Study 2

This study involves 14-day-pregnant C57BL/6 mice. NASH is established by a single subcutaneous injection of 200 μg streptozotocin (Sigma, USA) after birth and feeding with a high fat diet (HFD, 57% kcal fat, CLEA Japan, Japan) ad libitum after 4 weeks of age (day 28±2). Randomization of NASH mice into six groups of 12 mice at 6 weeks of age (day 42±2) and six groups of 12 mice at 9 weeks of age (day 63±2), the day before the start of treatment, respectively. NASH animals are dosed from age 6-9 weeks (Study 1), or from age 9-12 weeks (Study 2) with: vehicle, nidufexor, Compound 1, nidufexor+Compound 1. A non-disease vehicle-control group of 12 mice is included in both Study 1 and Study 2. These animals are fed with a normal diet (CE-2; CLEA Japan) ad libitum.

PK samples are collected and stored at ≤−60° C. Animals are dosed according to the dosing schedule below. Each animal is sacrificed 5 hours after last morning dose on the last day of study treatment.

Dosing:

Nidufexor is prepared in 0.5% (w/v) methylcellulose with 1% Tween® 80 in sterile water for injection (USP).

Compound 1 is prepared in 0.5% (w/v) methylcellulose (400 cPs) aqueous solution containing 0.5% (v/v) polysorbate 80, NF, in reverse osmosis water.

In general, vehicle, monotherapies, and combination therapy are administered once daily.

Measurements:

The following parameters are measured or monitored daily: individual body weight, survival, clinical signs and behavior of mice.

Pharmacokinetic measurements: PK samples are collected from 4 animals per time point per compound. PK samples for Compound 1 are taken at hours 1 and 24 on Days 6 and 10 (n=4 per time point) for both monotherapy and combination groups. Only one PK sample was collected per animal using the first 8 animals per group.

End of Treatment Measurements:

Mice are sacrificed at 9 weeks of age (study 1) or at 12 weeks of age (study 2). The 8 NASH animals that do not receive any treatment or vehicle are sacrificed at week 9 as a ‘baseline’ in order for comparisons of any fibrosis regression events observed in treated animals.

The following samples are collected: plasma, liver (fresh liver samples for gene expression analysis are collected at 5 hr post the last dose for each animal), stool. Organ weight is measured.

The following biochemical assays are performed: Non-fasting blood glucose in whole blood by Life Check (Eidia, Japan); serum ALT by FUJI DRI-CHEM (Fujifilm, Japan); serum triglyceride; serum MCP-1, RANTES (CCL5) and MIP-1α/MIP-1 quantification by a commercial ELISA kit; liver triglyceride by Triglyceride E-test kit (Wako, Japan); liver hydroxyproline quantification by hydrolysis method; histological analyses for liver section; HE staining and estimation of NAFLD Activity score; Sirius-red staining and estimation of fibrosis area (with and without perivascular space subtracted); oil red staining and estimation of fat deposition area; F4/80 immunohistochemistry staining and estimation of inflammation area; alpha-SMA immunohistochemistry staining and estimation of α-SMA positive area Gene expression assays using total RNA from the liver.

Real-time RT-PCR analyses are performed for: MCP-1, MIP-1α/β, RANTES, Emr1, CD68, TGF-β1, CCR2/5, TIMP-1, Cola1A1, TNF, IL-10, MMP-9, ≢-SMA and CX3CR1/CX3CL1, SHP (small heterodimer partner), BSEP (bile salt export pump), Cyp8b1.

Statistical tests are performed using one-way ANOVA followed by Dunnett's test and the Mann-Whitney test, as appropriate, for the multiple group comparisons. P values <0.05 are considered statistically significant.

### Example 7—Safety, Tolerability and Efficacy of Licogliflozin, an SGLT1/2 Inhibitor in Patients with Non-Alcoholic Fatty Liver Disease: Interim Analysis of a Placebo-Controlled, Randomized Phase 2a Study

A randomized, double blinded, placebo-controlled Phase 2a study was conducted to evaluate the safety, tolerability and efficacy of licogliflozin in patients with either histologically confirmed NASH or with a biochemical phenotype suggestive of NASH.

Method: Patients with histologically confirmed NASH (F1-F3) or phenotypic NASH (BMI≥27 kg/m² in non-Asians or ≥23 kg/m² in Asians, ALT≥50 (males) or ≥35 (females) and type 2 diabetes (T2DM)) received daily oral licogliflozin at 150 mg, 30 mg or placebo in a 2:2:1 ratio for 12 weeks (NCT03205150). The primary endpoint was the effect on ALT level after 12 weeks of treatment. Secondary endpoints include improvement in body weight, liver fat content and AST, amongst others. The study size was 110 of which 77 completed (placebo (n=18); licogliflozin 30 mg (n=25) and licogliflozin 150 mg (n=34)) and are included in the interim analysis.

Results: After 12 weeks of treatment, there was a 27% (17.2 U/L, p=0.036) and 19% (11.1 U/L, p=NS) placebo adjusted reduction from baseline levels of ALT at 150 mg and 30 mg, respectively. There was a reduction in AST of 30% (p=0.004) and 23% (p=0.043) as well as a 32% (p=0.001) and 26% (p=0.014) in GGT at 150 mg and 30 mg doses, respectively. Placebo adjusted reductions in body weight at both doses (˜4%, p=0.0001) and HbA1c (absolute change: 150 mg, 0.96% (p=0.0001); 30 mg, 0.81% (p=0.001)) were seen. Relative reduction in liver fat content was 22% (p=0.01) and 10% (p=NS) at 150 mg and 30 mg, respectively, and the proportion of patients with at least a 30% relative reduction was 66.7% (150 mg), 39.5% (30 mg) and 25% (placebo). Absolute reduction in liver fat was 4.45% (p=0.01) at 150 mg and 2.71% (p=NS) at 30 mg with 63.3% (150 mg), 43.5% (30 mg) and 18.8% (placebo) of patients achieving at least 5% absolute reduction. Diarrhea, the most common adverse event (AE), was reported by similar number of patients in the placebo and 30 mg group (38.9% vs. 40%) but was higher at the 150 mg dose (76.5%). Most diarrhea events (97.4%) were mild.

The study showed that licogliflozin is safe and tolerable and improves multiple biochemical endpoints associated with NASH after 12 weeks of treatment. The study achieved its primary end-point of statistically significant reduction in ALT of at least 25% compared to placebo as showed above (mean relative decrease in ALT of 27% and 19% versus placebo at 150 mg and 30 mg, respectively and statistically significant reductions in AST and GGT versus placebo at both doses).

The full analysis of a 12-week study also showed that licogliflozin treatment led to dose-dependent improvements in liver injury and metabolic biomarkers:

Treatment with licoliglozin at 30 mg and 150 mg resulted in placebo-adjusted reductions in serum ALT of 21% (P=0.061) and 32% (P=0.002), respectively, at Week 12. At Week 12, licogliflozin treatment also resulted in statistically significant placebo-adjusted reductions in serum AST (30 mg, 21% [P=0.024]; 150 mg, 32% [P<0.001]) and GGT (30 mg, 24% [P=0.008]; 150 mg, 36% [P<0.001];

Statistically significant reductions in body weight and waist circumference were observed with licogliflozin compared with placebo at Week 12; Favorable impact on glycemic control and insulin sensitivity with licogliflozin was also evident from improvements in HbA1c and HOMA-IR at Week 12;

Dose-dependent decreases in absolute and relative liver fat content were observed with 12 weeks of licogliflozin treatment.

### Example 8—Compound 1 Inhibits Profibrotic Gene Expression

The ability of Compound 1 to inhibit the expression of profibrotic genes and decrease biomarkers of fibrosis was measured in precision cut liver slices generated using cirrhotic liver tissues from NASH patient explants and from rodent models of liver fibrosis and NASH.

In precision cut liver slices from 5 cirrhotic NASH patient explants, Compound 1 significantly reduced gene expression of collagen, type 1, alpha 1 (COL1A1) by 39% and also reduced metalloproteinase inhibitor 1 (TIMP1) after two days of treatment (Error! Reference source not found.). Data are mean +/−standard deviation from the 5 cirrhotic NASH patients. DMSO was used as the solvent and utilized at a constant concentration (0.1%) across the different groups. ALK5 was used as a positive control. Compound 1 also significantly reduced the level of FBN-C (26%), a C-terminal fragment of fibronectin (Bager et al 2016) in culture media. PRO-C1 (34%), PRO-C3 (16%), and PRO-C4 (15%), fragments of the respective collagen subtypes (Leeming et al. 2010, Nielsen et al. 2013, Leeming et al. 2013), were similarly reduced in culture media with Compound 1 treatment but did not achieve statistical significance.

### Example 9—Antifibrotic Activity of Compound 1 in a Mouse Model of Liver Fibrosis

Antifibrotic activity of Compound 1 was established in an abbreviated, 3-week, murine CCl₄ model of liver fibrosis. CCl₄ is a hepatotoxin that when injected into mice results in liver fibrosis (Constandinou 2005). Compound 1 was dosed during the final week of injury.

Levels of phosphorylated SMAD3 (pSMAD3)/SMAD3 in the liver, a readout of active TGF-β signaling, were significantly reduced with all doses of Compound 1, demonstrating a reduction in TGF-β signaling. Gene expression analysis indicated a significant reduction in hepatic Col1a1, Col1a2, and Col3a1 expression with all doses of Compound 1. Hepatic OHP concentration was not significantly changed with all doses of Compound 1.

In summary, therapeutic treatment with Compound 1, significantly reduced levels of pSMAD3/SMAD3 in the liver, hepatic collagen gene expression and hepatic OHP concentration.

### Example 10—Antifibrotic Activity of Compound 1 in a Mouse Model of NASH

Compound 1 was also demonstrated to be an effective antifibrotic agent in the CDAHFD NASH mouse model. CDAHFD injury is a rodent model of NASH displaying liver fat accumulation, inflammation, and fibrosis (Matsumoto 2013). Three types of studies were performed: 1) prophylactic, Compound 1 in an abbreviated 3-week CDAHFD model; 2) therapeutic, Compound 1 for 6 weeks in the 11- to 12-week CDAHFD model; and 3) Compound 1 for 4 weeks in a 10-week CDAHFD model.

Compound 1 was tested prophylactically in an abbreviated 3-week CDAHFD model at low, medium and high doses across two independent studies. pSMAD3 levels in the liver were decreased by 19% at high dose, suggesting reduced activation of TGF-β. At high dose, Compound 1 significantly reduced hepatic OHP concentrations by ˜30% in both studies. Significantly reduced gene expression of collagens was observed in one of the studies at high dose and expression of Ehhadh, a gene for a peroxisomal bifunctional enzyme involved in fatty acid metabolism, was significantly elevated at high dose in both studies.

Compound 1 was tested therapeutically in 11- to 12-week CDAHFD injury studies at medium, high and highest doses across 3 independent studies. All doses significantly reduced hepatic OHP by up to 38% and pSMAD3 levels by up to 57%. Compound 1 also caused significant reduction in OHP concentration (24%). Collagen gene expression (Col1a1 and Col3a1) was significantly reduced at high and highest doses, as well as gene expression of profibrotic markers of connective tissue growth factor (Ctgf), matrix metalloproteinase 2 (Mmp-2), and Timp1. Gene expression of peroxisomal acyl-coenzyme A oxidase 1 (Acox1) and Ehhadh, which are involved in fatty acid metabolism, was significantly increased. Histological analysis of tissue showed a significant reduction in collagen area and the composite fibrosis score determined through second harmonic generation imaging indicated a significant reduction in quantity and quality of the collagen fibers.

In a 10-week CDAHFD study, the efficacy of Compound 1 was compared to the pan-αᵥ inhibitor CWHM12 (3S)—N-[3-hydroxy-5-[(1,4,5,6-tetrahydro-5-hydroxy-2-pyrimidinyl)amino]benzoyl] glycyl-3-[3-bromo-5-(1,1-dimethylethyl)phenyl]-β-alanine). pSMAD3 levels were reduced by 40% and 61% and OHP concentrations by 24% and 30% with Compound 1 and CWHM12, respectively. Although pan-αᵥ inhibition with CWHM12 reduced pSMAD and OHP levels, selective αᵥβ₁ inhibition was sufficient for antifibrotic activity.

In summary, treatment with Compound 1, a small molecule antagonist of αᵥβ₁, prophylactically or therapeutically, blocked SMAD3 phosphorylation and significantly decreased OHP levels, collagen gene expression, and collagen deposition examined histologically in the CDAHFD NASH mouse model. These findings were replicated in multiple studies.

### Example 11—First-In-Human Study of the Safety, Tolerability, PK, and PD of Compound 1

Part A (Single Ascending Dose Study)

Part A of the study was a first-in-human, randomized, double-blind, placebo-controlled, parallel-group, single ascending dose study of the safety, tolerability, and PK of Compound 1 in a maximum of 50 healthy male and female (non-childbearing potential) participants. Forty participants will be enrolled in up to 5 sequential cohorts.

Part B (Multiple Ascending Dose Study)

Part B of the study is on-going and was initiated after the first 2 cohorts in Part A of the study had been completed. The doses in Part B were determined from Part A of the study and were not higher than the highest dose that was administered in Part A of the study.

Part B of the study is a randomized, double-blind, placebo-controlled, parallel-group, multiple ascending dose study of the PK, PD, safety, and tolerability of Compound 1 administered for 7 days in a maximum of 40 healthy male and female (non-childbearing potential) participants.

Compound 1 is thus far well tolerated in all healthy volunteers.

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A pharmaceutical combination comprising 1) an α_{V}β₁ integrin inhibitor and 2) at least one additional therapeutic agent, for simultaneous, sequential, or separate administration.

2. The pharmaceutical combination of claim 1, wherein the α_{V}β₁ integrin inhibitor is (S)-2-(4-methyltetrahydro-2H-pyran-4-carboxamido)-9-(5,6,7,8-tetrahydro-1,8-naphthyridin-2-yl)nonanoic acid, a stereoisomer, a tautomer, an enantiomer, a pharmaceutically acceptable salt, a prodrug, an ester, or an amino acid conjugate thereof.

3. The pharmaceutical combination of claim 1 or 2, wherein the at least one additional therapeutic agent is selected from the group consisting of: a FXR agonist (e.g. M480 (Metacrine), NTX-023-1 (Ardelyx), INV-33 (Innovimmune), and obeticholic acid), Steroyl-CoA desaturase-1 (SCD-1) inhibitor (e.g., arachidyl amido cholanoic acid (Aramchol™)), THR-β agonist (e.g., MGL-3196 (Resmetirom), VK-2809, MGL-3745 (Madrigal)), galectin-2 inhibitor (e.g., GR-MD-02/Belapectin), PPAR agonist (e.g., saroglitazar, seladelpar, elafibranor, lanifibranor, lobeglitazone, pioglitazone, IVA337 (Inventiva), CER-002 (Cerenis), MBX-8025 (Seladelpar)), GLP-1 agonist (e.g., exenatide, liraglutide, semaglutide, NC-101 (Naia Metabolic), G-49 (Astrazeneca), ZP2929 (BI/Zealand), PB-718 (Peg Bio)), FGF agonist (e.g., pegbelfermin (ARX618), BMS-986171, NGM-282, NGM-313, YH25724, and proteins disclosed in WO2013049247, WO2017021893 and WO2018146594), tirzepatide, pyruvate synthase inhibitors (e.g., nitazoxanide), Apoptosis signal-regulating kinase 1 (ASK1) inhibitor (e.g., selonsertib (GS-4997), GS-444217), Acetyl-CoA carboxylase (ACC) inhibitor (e.g., firsocostat (GS-0976), PF-05221304, gemcabene (Gemphire)), CCR inhibitor (e.g., AD-114 (AdAlta), Bertilimumab (Immune), CM-101 (ChemomAb), CCX-872 (ChemoCentryx), Cenicriviroc), thiazolidinedione (e.g, MSDC-0602K, Pioglitazone), sodium-glucose co-transporter-2 and 1 (SGLT1/2) inhibitor (e.g., Remogliflozin, luseogliflozin, dapagliflozin, licogliflozin), DPP-4 inhibitor (sitagliptin, saxagliptin, vildagliptin, linagliptin, evogliptin, gemigliptin, anagliptin, teneligliptin, alogliptin, trelagliptin, omarigliptin, gosogliptin, dutogliption), insulin receptor agonist (e.g. ORMD 0801 (Oramed)), SGLT-2 inhibitor with DPPP inhibitor (e.g. empagliflozin and linagliptin), insulin sensitizer (e.g., MSDC-0602K (Octeta/Cirius)), CCR2/5 inhibitor (e.g., CVC (Allergan), anti-BMP9 antibodies (e.g., the antibodies described in WO2016193872); a compound selected from the group consisting of ((R)-3-amino-4-(5-(4-((5-chloropyridin-2-yl)oxy)phenyl)-2H-tetrazol-2-yl)butanoic acid; (R)-3-amino-4-(5-(4-((5-chloro-3-fluoropyridin-2-yl)oxy)phenyl)-2H-tetrazol-2-yl)butanoic acid; (R)-3-amino-4-(5-(3-((5-(trifluoro-methyl)pyridin-2-yl)oxy)phenyl)-2H-tetrazol-2-yl)butanoic acid; (R)-3-amino-4-(5-(4-((5-(trifluoro-methyl)pyridin-2-yl)oxy)phenyl)-2H-tetrazol-2-yl)butanoic acid; (S)-3-amino-4-(5-(4-((5-chloro-3-fluoropyridin-2-yl)oxy)phenyl)-2H-tetrazol-2-yl)butanoic acid; (R)-3-amino-4-(5-(4-phenethoxyphenyl)-2H-tetrazol-2-yl)butanoic acid; and (R)-3-amino-4-(5-(4-(4-chlorophenoxy)-phenyl)-2H-tetrazol-2-yl)butanoic acid;
or a pharmaceutically acceptable salt thereof, or any combination thereof.

4. The pharmaceutical combination of any of claims 1 to 3, wherein the one additional therapeutic agent is the sodium-glucose co-transporter (SGLT) inhibitor, e.g. sodium-glucose co-transporter-2 and 1 (SGLT1/2) inhibitor.

5. The pharmaceutical combination of claim 4, wherein the SGLT inhibitor is selected from: licogliflozin, dapagliflozin, canagliflozin, empagliflozin, ipragliflozin, ertugliflozin, mizagliflozin.

6. The pharmaceutical combination of any of claims 1 to 5, wherein the pharmaceutical combination is a fixed dose combination.

7. The pharmaceutical combination of any of claims 1 to 5, wherein the pharmaceutical combination is a free combination.

8. Use of the pharmaceutical combination of any one of claims 1 to 7, in the manufacture of a medicament for preventing, delaying or treating a liver disease or disorder.

9. The use of claim 8, wherein the liver disease or disorder is a fibrotic or cirrhotic liver disease or disorder.

10. A method of preventing, delaying or treating a liver disease or disorder, in a patient in need therefor, comprising administering a therapeutically effective amount of the pharmaceutical combination of any of claims 1 to 7.

11. The method of claim 10, wherein the liver disease or disorder is a fibrotic or cirrhotic liver disease or disorder, selected from the group consisting of non-alcoholic fatty liver disease (NAFLD), non-alcoholic steatohepatitis (NASH), liver cirrhosis, alcohol-induced cirrhosis, cystic fibrosis-associated liver disease (CFLD), liver fibrosis, and progressive fibrosis of the liver caused by any of the diseases above or by infectious hepatitis.

12. The method of claim 10, wherein the liver disease or disorder is non-alcoholic fatty liver disease (NAFLD), non-alcoholic steatohepatitis (NASH), liver fibrosis, or liver cirrhosis.
